(19) United States
(12) Patent Application Publication
(10) Pub. No.: US 2024/0387044 A1
(43) Pub. Date: Nov. 21, 2024

(54) SYSTEMS AND METHODS FOR IMPROVING PATIENT OUTCOMES FOR MUSCULOSKELETAL CARE

(71) Applicant: MSKai, LLC, Memphis, TN (US)

(72) Inventors: Sreenivas Sukumar, Seattle, WA (US); Adam Bruggeman, San Antonio, TX (US); John Burrow, Spotsylvania, VA (US); Lloyd Wade, Oxford, MS (US); Larry Roberts, Cookeville, TN (US)

(21) Appl. No.: 18/664,030

(22) Filed: May 14, 2024

Related U.S. Application Data
(60) Provisional application No. 63/502,145, filed on May 15, 2023.

(51) Int. Cl. G16H 50/20; G16H 10/60; G16H 15/00; G16H 30/40

(52) U.S. Cl. CPC G16H 50/20; G16H 10/60; G16H 15/00; G16H 30/40

(57) ABSTRACT

A system for improving patient outcomes for musculoskeletal care that utilizes an algorithm trained using images of musculoskeletal pathology. The system can include numerous models where each model can be trained with images from MRIs, CT Scans, X-Rays, and PET scans, or any other existing imaging technology capable of imaging a patient's musculoskeletal pathology. The system can annotate the images to generate reports with the annotations for further use in diagnosing a disease or reporting to an insurance provider.

### FIELD

This disclosure relates to the improvement of treatment for patients with musculoskeletal issues by implementing systems enhanced with artificial intelligence and machine learning.

### BACKGROUND

Radiological imaging delays have been shown to be an independent predictor of length of hospital stay and, in the case of CT and MRI, increased hospital episode costs. Additionally, radiological imaging can also be a diagnostic gatekeeper in terms of the initiation of treatment pathways, especially when greater than 75% of clinical decisions are based on medical imaging. Furthermore, Radiologist shortages are being observed, with the severity of the shortage set to increase. This growing shortage is being compounded with the issues around delays in treatments caused by the challenges arising from the requirements by insurance companies for prior authorizations.

Prior authorizations for healthcare services exist to ensure patients receive only those services that are medically necessary and fall within policy guidelines. Without such safeguards, the economic viability (and actuarial assumptions) of health insurance becomes impaired, causing expenditures to exceed the insurance premiums and compromising an insurer's long-term viability to serve its constituents. However, the coverage-determination process has become exceedingly expensive, effort-intensive, and operationally inefficient, resulting in delayed implementation of patient care and treatment. This can be problematic for providers; according to a 2020 review of 30,000 orthopedic orders, 97% were approved without any denial in the authorization process, and another 2% were ultimately approved after re-examination (99% approval process). However, a physician's office has to maintain its infrastructure to track all order sets in case of escalation, with 17% of authorizations requiring additional clinical data; this can add 7 days to the authorization process. Compliance with the coverage-determination process represents a deadweight loss to the healthcare ecosystem. This has led to tremendous resources being expended to verify facts, perhaps more resources than determining the existence of the covered pathology. Without objective, consistent data facilitating unbiased, accurate conclusions/diagnoses and then facilitating appropriate treatment pathways, this paradigm will persist.

On the physician side, medical assistants manage prior-authorization processes as an administrative function. These assistants do not have the requisite knowledge to “advocate” for a treatment pathway/surgery. If physicians were to assume primary responsibility for these processes and approvals, physician capacity would fall, and healthcare costs would rise. Data confirms physician shortages within the US already, without considering this added inefficiency. On the insurer side, nurses usually manage the prior-authorization process. Insurers are employing few board-certified radiologists or surgeons because of the cost of primary-care physicians not being appropriately trained. Radiologist trends relative to demographics continue to weaken, limiting processing capacity. Underlying this entire process, policy eligibility is a dynamic construct, constantly in flux, adding time for prior authorization approval as an explicit prolonging factor. However, implicitly, changing policy requirements delays treatment because physicians are not aware of the change and do not highlight the most relevant details for the currently active policy (oftentimes, the right data is not collected from the patient to satisfy new requirements). In some cases, transparency/insight into policy requirements are inaccessible.

There are tactical inefficiencies for the physician in the current environment. Radiologists have traditionally been thought to be independent interpreters of imaging who have no loyalty to the ordering physician or the payor. As such, radiologists are not incentivized to be accommodating to insurers/payors. Payment is constant, no matter whether the findings lead to surgery or denial of treatment. However, the risk of “underinterpreting” is real. Longer reports do not pay more. The lower bound is an omission creating a malpractice claim. Concern exists about whether radiologists are lobbied by surgeons on interpretations and that surgeons may direct future radiological business to “more friendly” radiologists. This inconsistency is challenging to the actuarial efforts of the insurer from a predictive perspective and ultimately to profitability.

Furthermore, radiologists are not aware of payor requirements for approval. Surgeons often re-engage radiologists for increased precision (for qualification by payor's metrics) or to request slight changes of language to fit payor requirements. Variance has been found/proven in radiological reads across radiologists and longitudinally for the same radiologist. This finding implies that who reads the report may be as important as what the images reveal. Ultimately, these dynamics suggest, potentially, that neither ordering physician nor payor can have full confidence in the radiologist's report. Improvements are needed in musculoskeletal care for better patient outcomes.

### SUMMARY

According to this disclosure, an embodiment of a method for improving identification of pathologies for musculoskeletal patients can comprise the following steps: first, providing a computing unit configured to utilize an algorithm trained with an AI engine using images of musculoskeletal pathology, followed by the obtaining of at least one patient image, and then applying the algorithm to the patient image to identify a pathology within the image. This can be followed by applying an application stored within the computing unit to annotate anatomical measurements within the algorithmically analyzed patient image, which may then generate a report comprising an annotated patient image.

In another embodiment of the presently disclosed method for improving identification of pathologies for musculoskeletal patients, the AI engine may be either a knowledge-centric intelligence model or a neural network model. In a variation of the presently disclosed method, the at least one patient image may be obtained via a further step following the analysis of the at least one patient image by a modality detector. In a different variation of the embodiment, a decision from the neural network model may be validated using a feature-measurement model.

In another embodiment of the presently disclosed method, the knowledge-centric intelligence model can be configured to generate an explanation of a decision by the algorithm. In a further embodiment of the method for improving pathologies for musculoskeletal patients, a 3D model may be used to overlay physics-based measurements and algorithmic predictions onto the image.

In a separate version of the method embodiment disclosed above, after the at least one patient image is analyzed by the modality detector, there may be a further step that follows where an analysis of the at least one patient image can occur with at least one secondary model.

In an embodiment of a system for generating annotated patient images there can be a computing unit configured to utilize an algorithm that can be trained with an AI engine using images of musculoskeletal pathology. In such a system, there can be at least one patient image that the algorithm can be applied to, to identify a pathology within the patient image. Such a system may further contain an application, which can be stored within the computing unit, and where the application can be configured to annotate anatomical measurements within the algorithmically analyzed patient image. The system may further use the computing unit, which can be configured to generate a report comprising the annotated patient image.

A variation of the above systems, the AI engine may be either a knowledge-centric intelligence model or a neural network model. In a separate variation of the above system, the computing unit may further comprise a modality detector that can be configured to analyze the at least one patient image.

Furthermore, the above system may be configured to use the knowledge-centric intelligence model that can be configured to generate an explanation of a decision by the algorithm, where the explanation can be added to the report. A variation of such a system can include the computing unit, which may further comprise a feature-measurement model, where the feature-measurement model can be configured to validate a decision from the neural network model.

Such a system may further include a 3D model that can output an overlay of physics-based measurements and algorithmic predictions.

In another variation of the above system, there may be at least one secondary model, where the at least one secondary model can be configured to receive and analyze the at least one patient image from the modality detector.

In a separate variation of the above system, the report can be provided via a web-based application.

The above summary is not intended to describe each and every example or every implementation of the disclosure. The description that follows more particularly exemplifies various illustrative embodiments.

### BRIEF DESCRIPTION OF THE DRAWINGS

The following description should be read with reference to the drawings. The drawings, which are not necessarily to scale, depict examples and are not intended to limit the scope of the disclosure. The disclosure may be more completely understood in consideration of the following description with respect to various examples in connection with the accompanying drawings.

FIG. 1 is a flow chart of an embodiment of the systems and methods for improving patient outcomes for musculoskeletal care.

FIG. 2 is a partial flow chart of an embodiment of the systems and methods for improving patient outcomes for musculoskeletal care.

FIG. 3 is a partial flow chart of an embodiment of the systems and methods for improving patient outcomes for musculoskeletal care.

FIG. 4 is a flow chart for an embodiment of the data ingestion for generating a data model repository.

FIG. 5 is a flow chart of an embodiment of the AI engine development for the systems and methods for improving patient outcomes for musculoskeletal care.

FIG. 6 is an overview flow chart of an embodiment of the systems and methods for improving patient outcomes for musculoskeletal care.

FIG. 7 is an overview flow chart of an embodiment of the AI engine development for the systems and methods for improving patient outcomes for musculoskeletal care.

FIG. 8 is an overview flow chart of an embodiment of presentation tools for the systems and methods for improving patient outcomes for musculoskeletal care.

FIG. 9 is Flow Diagram 1, an overview of a process for registering users in a web-based application.

FIG. 10 is Flow Diagram 2, an overview of the process for a user login to a web-based application.

FIG. 11 is Flow Diagram 3, an overview of a process for a user needing login support in a web-based application.

FIG. 12 is Flow Diagram 4, an overview of a process for a user accessing patient images in a web-based application.

FIG. 13 is Flow Diagram 5, an overview of a process for a web-based application analyzing patient images.

FIG. 14 is Flow Diagram 6, an overview of a process for a web-based application generating reports on analyzed patient images.

FIG. 15 is an example of an annotated image created by an embodiment of the web-based application.

FIG. 16 is an example of an annotated image created by an embodiment of the web-based application.

FIG. 17 is an example of an annotated image created by an embodiment of the web-based application.

FIG. 18 is an example of an annotated image created by an embodiment of the web-based application.

FIG. 19A is an example of an annotated image created by an embodiment of the web-based application.

FIG. 19B is an example of an annotated image created by an embodiment of the web-based application.

FIG. 19C is an example of an annotated image created by an embodiment of the web-based application.

FIG. 19D is an example of an annotated image created by an embodiment of the web-based application.

### DETAILED DESCRIPTION

The present disclosure relates to systems and methods for improving patient outcomes for musculoskeletal care. Various embodiments are described in detail with reference to the drawings, in which reference numerals may be used to represent parts and assemblies throughout the several views. References to various embodiments do not limit the scope of the systems and methods disclosed herein. Examples of construction, dimensions, and materials may be illustrated for the various elements; those skilled in the art will recognize that many of the examples provided have suitable alternatives that may be utilized. Any examples set forth in this specification are not intended to be limiting and merely set forth some of the many possible embodiments for the systems and methods. It is understood that various omissions and substitutions of equivalents are contemplated as circumstances may suggest or render expedient. Still, these are intended to cover applications or embodiments without departing from the disclosure's spirit or scope. Also, it is to be understood that the phraseology and terminology used herein are for the purpose of description and should not be regarded as limiting.

In an embodiment of a system for improving patient outcomes for musculoskeletal care, there can be the creation of a model playbook, described infra, which may create a process for spinal image processing for diagnostics and decision-making purposes. The system may include AI engines for the analysis of sets of images acquired from medical diagnostic imaging systems, such as X-ray, MRI, and CT. The MRI images may consist of cervical, thoracic, and lumbar arrangements. Other modalities may also be used to acquire a set of medical images of a patient. The sets of images may consist of a variety of different views, e.g., sagittal, axial, or coronal views. The AI engines can be configured to determine slice-selection, view identification, modality detection, manual, automatic, and semi-automatic labeling, semantic segmentation, anatomical annotation, pathology diagnosis, extraction of measurements, image-based report generation, and medical procedure recommendation. The system may further be configured to use the AI engines to determine pathologies contained within a set of acquired images from a patient suffering from said pathologies.

The approach being taken for the development of the models is knowledge-centric intelligence, a form of AI designed to capture the knowledge of human experts to support decision-making. The creation of the AI engine models are based on the disclosures provided in Sukumar et al. U.S. Pat. No. 10,127,292 and Potok et al. U.S. Pat. No. 10,643,031 herein fully incorporated by reference. However, such information was a mere starting point for the embodiments contained herein. In the workflow in some of the embodiments of this invention the knowledge of experts and specialists can be digitized for utilization by the AI engine. For example, experts and specialists knowledge, such as radiologists (i.e., capture their perceptual/visual model of the spine), billing specialists (i.e., capture their knowledge of insurance authorization and payments), insurance agents (i.e., capture their rule-base of beneficiary eligibility and coverage) and orthopedists (i.e., capture their physical/mechanical understanding of the spine function) can be converted into mathematical/computational models. Further development of a decision framework specific to spine image analysis can be created, which may know how to orchestrate the digitized models of human knowledge representations in a workflow when posed with different modalities and views. Within an embodiment of a decision framework, a rule-based representation model for billing specialists and insurance agents, a neural network model for radiologists, and an equation-based model for orthopedists may be the preferred choice of a knowledge representation. Each of these models can be trained on a statistically significant number of data samples, capturing the combined community knowledge of radiologists, orthopedists, and insurance and billing agents.

Unlike the current practice of building data-driven systems (databases and data lakes) and model-driven AI (such as IBM Watson, ChaptGPT, etc.), embodiments of this invention can accommodate domain-specific knowledge. For example, the knowledgebase for spine-image representation and interpretation is described by the medical guidelines for the standard of care, the physics of the normal spine (i.e., its flexibility, agility, stress, shear, range-of-motion), or other known knowledgebase datasets. By design, the assumption is to accommodate guidelines that can change from year to year, and the accuracy of the digital approximation to the physics of the spine can evolve from modality to modality. However, embodiments of this system may have to automatically learn to adapt to new guidelines, rules, and representations. Such decision frameworks can be implemented such that when new “knowledge” is added to the framework (e.g., change in billing process, new imaging modality), models may then retrain themselves and adapt. Such a design can enable a seamless expansion to new skills in the future (e.g., handling higher resolution, diagnosing new types of injury, etc.) and thereby automation of the workflow.

Embodiments of the proposed system and methodology may operate with the following definition of explainability: “a user can ask for an explanation behind every algorithmic decision,” which can be in compliance with the “right of explanation” described in the European Union's General Data Protection Regulation (GDPR).

Such compliance can satisfy the explainability requirement in the following ways. First, embodiments of the models in AI are mathematically considered intrinsically explainable, i.e., they can be a linear regression, logistic regression, decision rules, decision-tree-based methods, random forests, generalized linear models, nearest neighbor methods, and Bayes-classifiers. Every algorithmic decision from our proposed methodology is first made using one of these explainable models.

Second, when such explainable models do not have the complexity to learn complex structures and patterns or do not satisfy the expectations of accuracy, precision, and recall, then a neural network model may be trained. Neural network models have the ability to capture patterns, features, and trends in data with increasing model parameter complexity-however, they suffer from being a “black box” and a lack of explainability. For example, pathology prediction using neural networks on images can be fast and accurate but not explainable. In such situations, a model may be created to validate the neural network inference using a human-like approach based on measurements and features. Only if the decision comes from the feature extraction/measurement approach is the model prediction/decision revealed to the user. In cases where there is no agreement between the neural network model and the feature extraction model, both results are presented for review by a human expert.

Third, a human expert can then dive deeper into every “algorithmic” output in the workflow—and interactively iterate with data, make corrections to algorithmic outputs, and override algorithmic errors or overtures. The human expert can be provided with one or more of the following explainability measurements:
partial dependence, individual conditional expectation, and accumulated local effect plots
feature interaction and importance rankings
Shapley values (SHAP), Local and global surrogates (LIME)
counterfactuals, adversarial examples

For image-based predictions and analysis, physics-based 3D models may serve as a baseline. Users can be shown overlays of the physics-based measurements and algorithmic predictions for inspection. The 3D model can be built from a huge database of normal spine images. If the segmentation and measurements are far off visually from the deep-learning/neural network model predictions, the algorithmic decision is not revealed until human verification of segmentation and labeling.

By following a strict decision-reveal paradigm as described above, a guarantee can be provided that every algorithmic decision will either come from an explainable mathematical model or human verification of the algorithm decision.

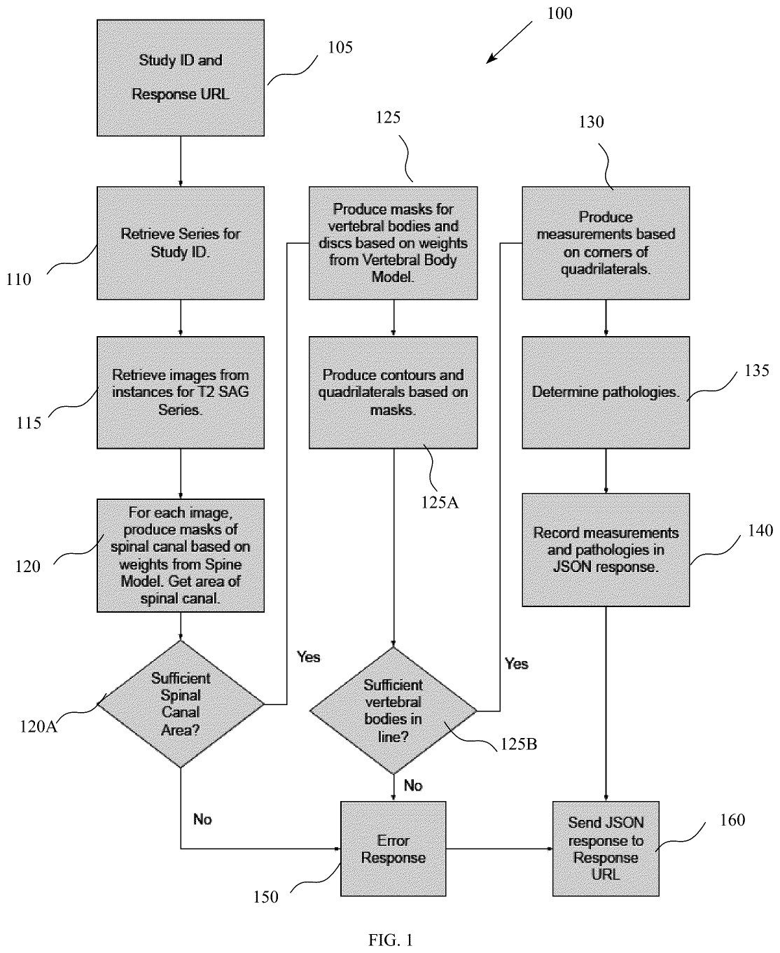

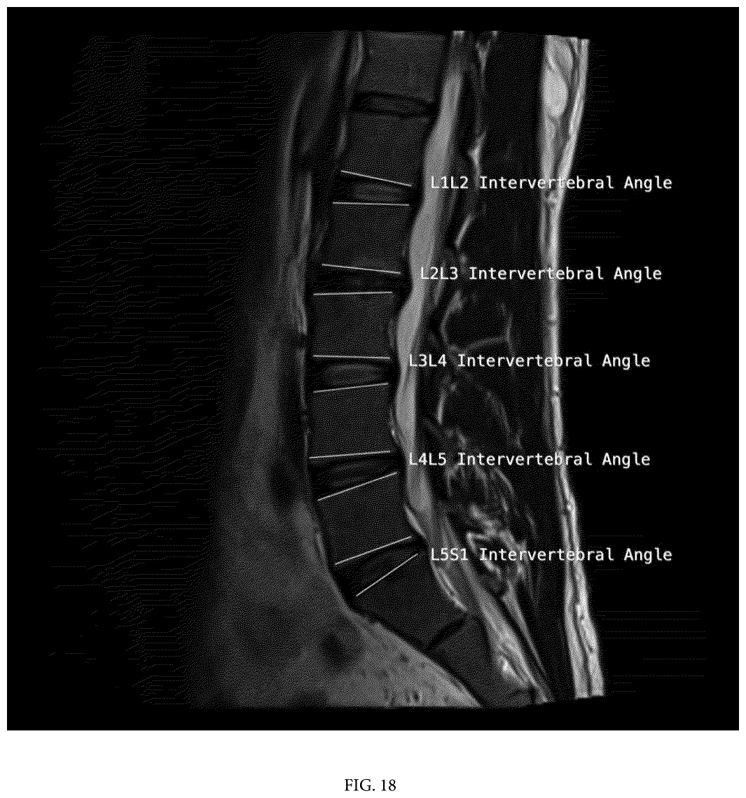

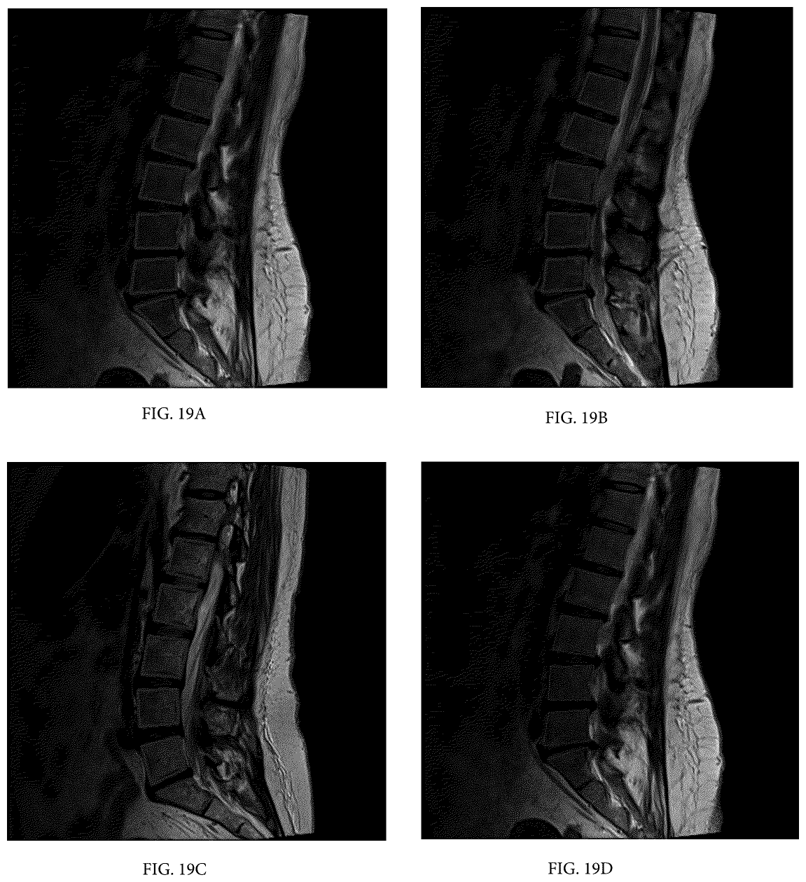

Referring to FIG. 1., in this flow chart, 100 is a breakdown of an embodiment of a method of analysis. Beginning with acquiring a study ID 105 for a patient, followed by retrieving images 110 associated with the series for the study ID. Once the images have been retrieved, any T2 SAG images within the series are then retrieved 115. From there, each image can be used to produce 120 masks of the spinal canal based on weights from the Spine Model; additionally, the area of the spinal canal is then calculated. The next step is the determination 120A of sufficient spinal canal area. In this embodiment, if the model determines that there is a sufficient spinal canal area, then the process can continue with the annotation of the images 125 by producing masks for vertebral bodies and discs based on weights from a Vertebral Body Model. If there is a determination 120A that there is not a sufficient spinal canal area an error 150 can occur, which may then require a new series of images. Once the images are annotated via the Vertebral Body Model, this embodiment can then further annotate the images 125A with contours and quadrilaterals based on masks; if there are no vertebral bodies in line, an error 150 can occur, which may then require a new series of images. Examples of the images with contours and quadrilaterals applied are illustrated in FIGS. 15-19A-D. FIG. 15 provides an example of the contours and quadrilaterals with applied labels while in FIGS. 19A-D, there are several examples of applied quadrilaterals on the vertebral bodies. With the addition of the contours and quadrilaterals, the production of measurements 130 can occur based on the corners of quadrilaterals.

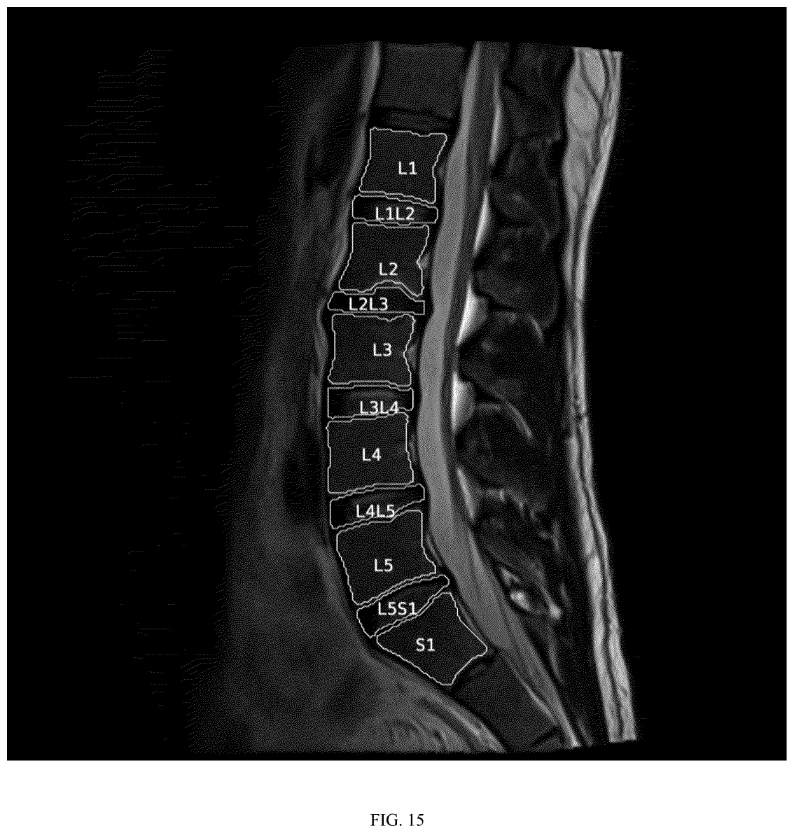

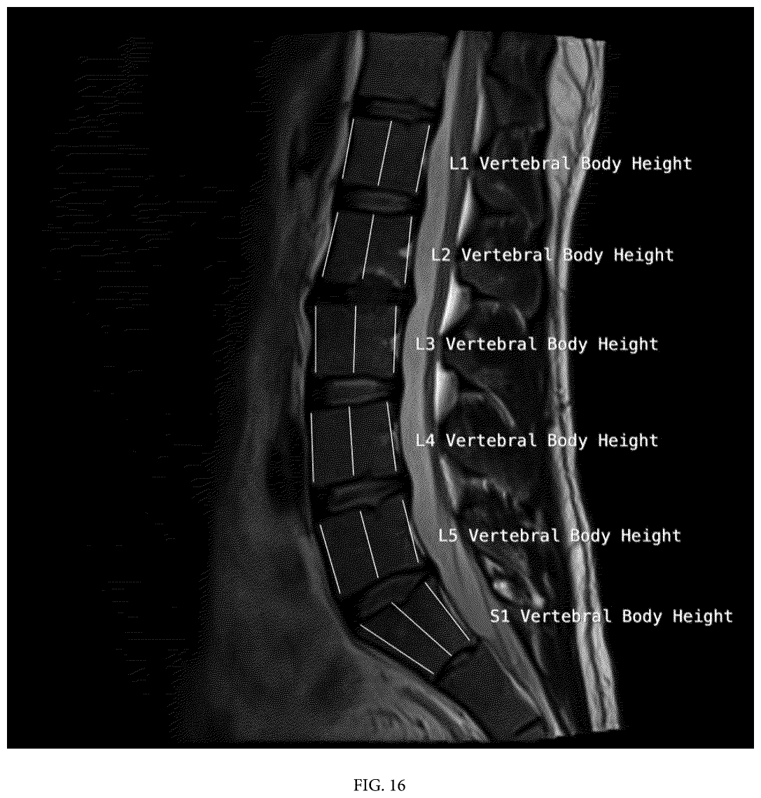

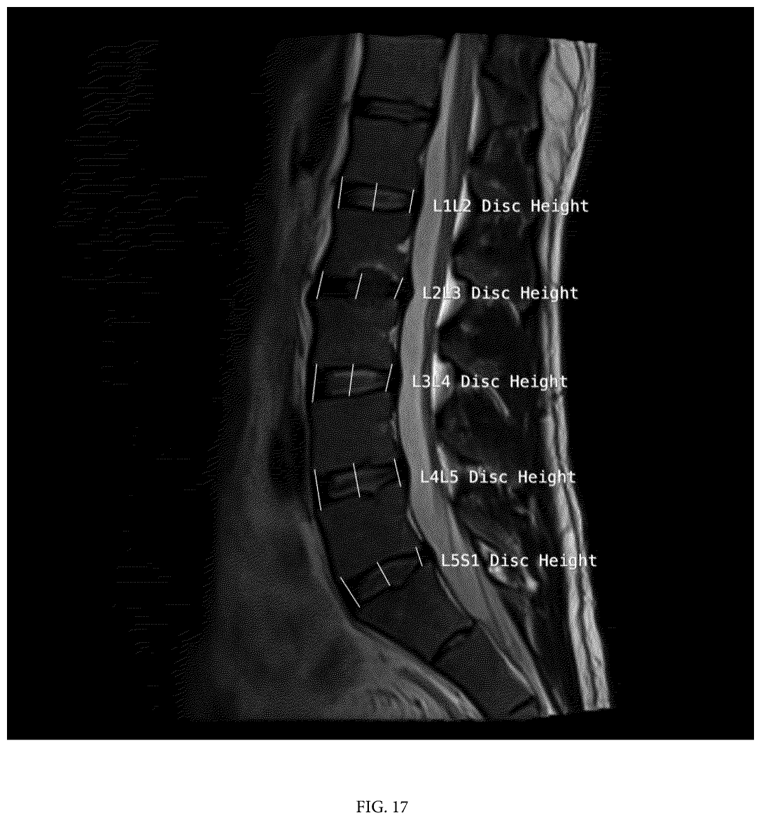

Examples of the images where measurements have been annotated are illustrated in FIGS. 16-18. In FIG. 16, there are examples of measurements of vertebral body height. FIG. 17 provides an example of disc height. While in FIG. 18, there is an example of the measurement for intervertebral angles. Once measured, the method of analysis 100 can then determine 135 pathologies within the patient's image series. From there, the measurements can be recorded along with the detected pathologies in a JSON response 140. Errors 150 or the JSON response 140 can then be sent as a response URL 160.

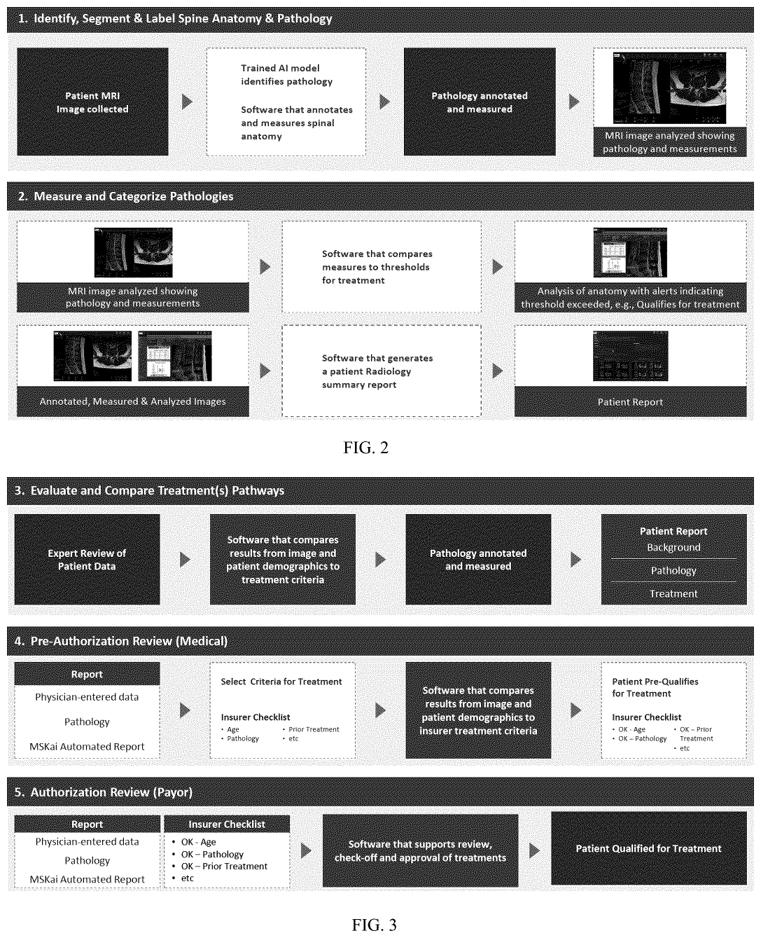

FIGS. 2 and 3 provide an example of an embodiment for training a model for inclusion within a model playbook. Such training is described in detail infra. FIG. 3 provides an overview of the stages for identifying pathologies from a set of patient-acquired images and the subsequent treatment and authorization steps of an embodiment of the systems and methods for improving patient outcomes for musculoskeletal care.

It has been contemplated that some embodiments may include input from insurance companies, where a representative of an insurance company can be a user. In such an embodiment, the insurer user may then set approval thresholds, treatment criteria, and validation checks. For example, in FIG. 2, a threshold can be set by a user; in this example, the insurer is the user.

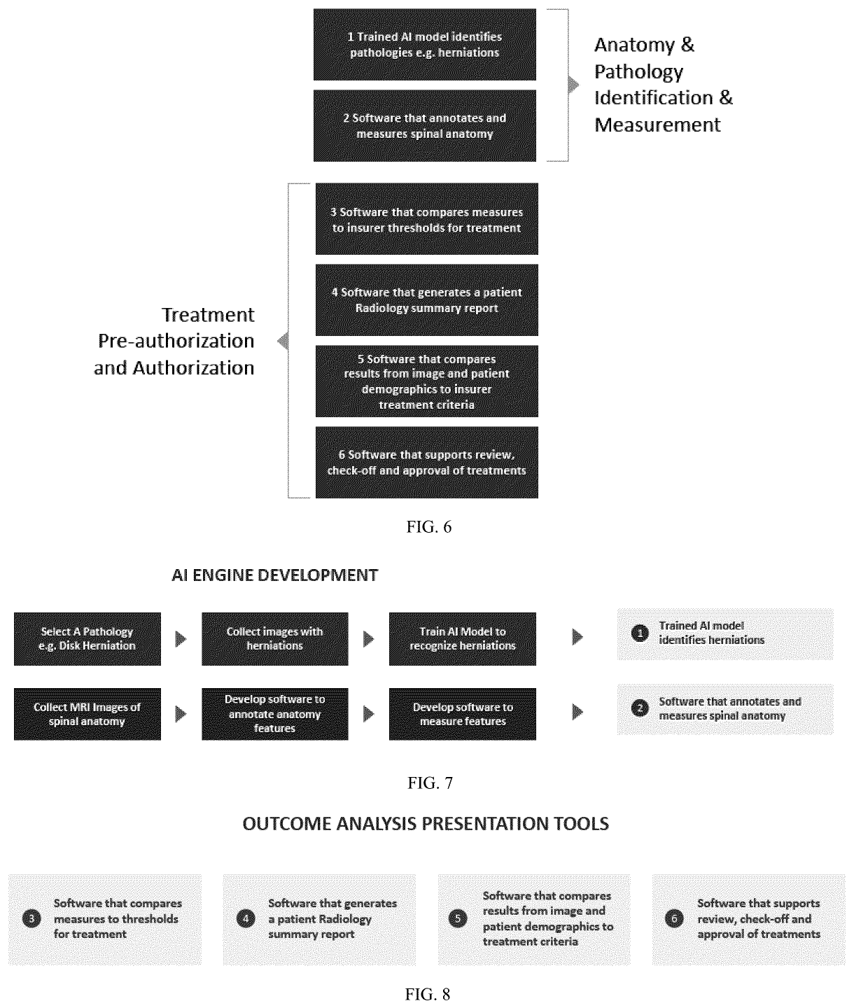

FIG. 6 provides an illustrative example of a training embodiment for a model's AI engine development.

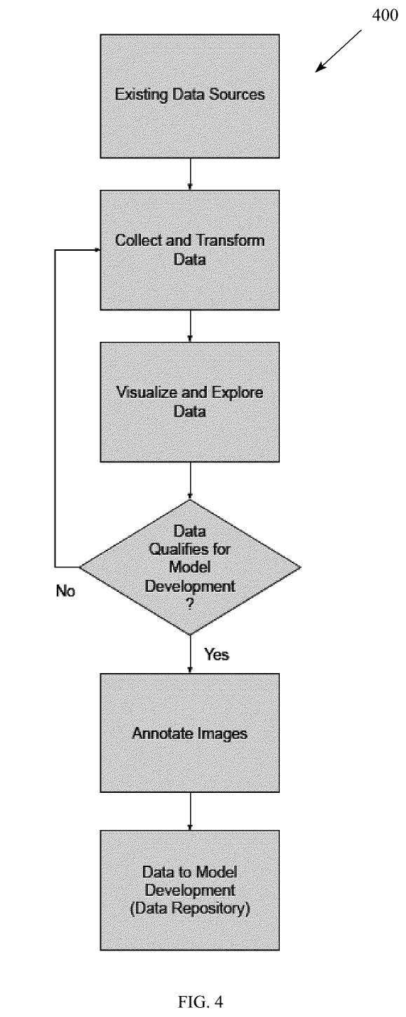

Regarding FIG. 4, is a general overview of an embodiment of a data ingestion process for the generation of a data repository that may be used in creating a model playbook. In the data ingestion process 400, first existing data sources are identified and acquired; for example, X-ray, CT, and MRI images from healthy and diseased individuals. The data is collected and transformed so that the images can be annotated and stored in the data repository for use in a model playbook creation.

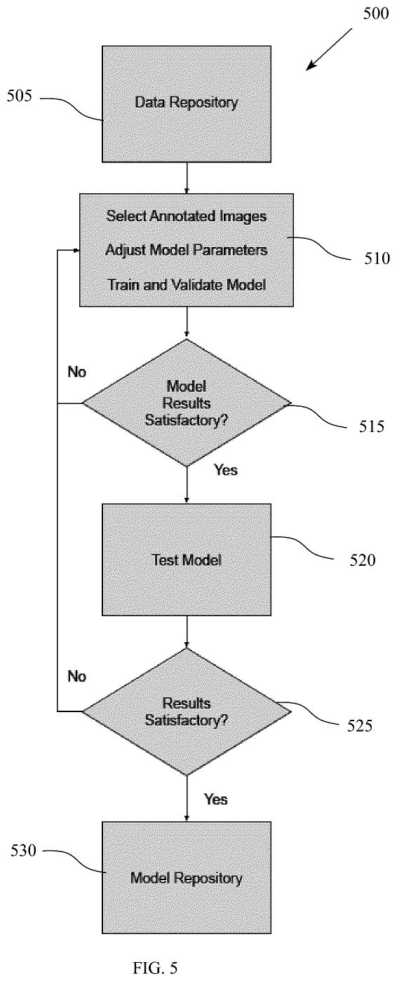

FIG. 5 provides a general overview of an embodiment of the model playbook. Model playbooks can be developed based on a curated ground truth dataset of patient imaging, annotation, and diagnosis data. Where the models within a model playbook can be trained to a desired accuracy and can be designed to capture perceptual knowledge of spinal imagery, structural and anatomy of healthy spines vs. injured ones, extract measurements from spinal images, and may have the ability to articulate measurements into natural language reports. The image in FIG. 5 is an embodiment of a process that may be used to train each model within the model playbook. The process 500 in FIG. 5 can begin with accessing a data repository 505, which may be followed by step 510, where annotated images can be selected, model parameters can be adjusted, and the model can be trained and validated. The following step 515 can be the determination of satisfactory results from the training and validation. If the training was successful, then the step 520 of testing the model can occur, followed by the step 525 of determining if the test results were successful. If, in step 515, the results were deemed unsatisfactory, then the process returns to step 510. If successful, step 530 can occur, where the validated model is added to a model repository. If the results in step 525 were deemed unsatisfactory, then the process returns to step 510. All of the following models can be developed using the described protocol of process 500.

The input data can be different, and the output predictor variables may also differ, but the process can be similar. Other models known by persons of skill in the art may also be included within the architecture of a model playbook. The following descriptions are some of the model embodiments that may be used within the model playbook.

In an embodiment that uses a Slice Selector model, where an application can utilize spinal images, patient data from an imaging source, modality, or a PACS archival system can be made available as a DICOM file; for example, a Spine Model. The file can consist of a series of image slices of the spine from a specific view. For e.g., 12 slices in the lumbar axial view 10 slices in the sagittal (SAG) view. Depending on the patient's size, age, height, and other medical conditions, not all slices are as informative—i.e., the disease pathology is not detectable in every image. The AI models can be trained to identify and select DICOM slices that may provide the best opportunity for an accurate diagnosis. The process for creating the slice detection model can take a DICOM image sequence as input, the index of the image in that sequence, where a human observer may have picked to annotate as the label, and then train an AI model with thousands (or more) of DICOM sequences. Over time, this model may automatically be able to pick the image a human expert would have picked for making the diagnosis.

In an embodiment that uses a modality detector model, in practice, such a model can use imaging that is done through multiple modalities. X-ray, CT, and MRI are a few examples. The perceptual task of interpreting disease conditions changes from modality to modality—i.e., a model trained for detecting X-rays will not work effectively on an MRI image. Therefore, a modality detection model can be created so that the right perceptual knowledge model can be applied to the right modality of the data. The modality detector model takes one or more slices of the DICOM file as input, associates the modality description with labels, and trains to automatically classify the modality as X-ray, CT, MRI, or any other known imaging modality. While it is trivial for humans to do this, this detector is critical for computers to automatically know which knowledge model to apply for the appropriate modality. With the use of a modality detector, the system for improving patient outcomes for musculoskeletal care can be malleable with a source of provided images.

Following the detection of a modality, secondary models may be applied to a patient image to further refine the analysis. For Example, a View Detector Model can be applied. In such a model, the detector may be able to automatically identify the view/perspective of the patient's image of the spine. In spinal imagery practice, the patient's spines are imaged from axial, sagittal, and coronal views. Some pathologies are revealed in some views better than others. The perceptual knowledge model of what to look for in an axial image is quite different from the sagittal image. The view detector model is trained to take any DICOM file as input and automatically recognize the view (axial, sagittal, coronal) captured by the image.

Another secondary model that may be applied to a patient image to further refine the analysis can be a Labeling Model. In this model, there can be the ability to segment the different parts of the spinal image (e.g., L1, L2, L3 . . . ). The model can be trained so that it takes the ground truth annotations of normal and disease spine annotations to propose possible segmentations of the different components. In other words, the labeling model can group spatially coherent pixels into regions of interest. Another secondary model can be a Semantic Segmentation Model, which may associate labels described above as L1, L2, and L3 based on the segmentation output from the labeling model; see FIG. 15 for an example of such labels.

A further secondary model can be a Anatomical Segmentation Model, which may be able to identify and mark the different parts of the spine-discs, canal, etc. For example, in a Vertebral Body Model as described in the method of analysis 100.

An additional secondary model that may be applied to a patient image to further refine the analysis can be an Explainable Measurements Model, which may be able to automatically extract distances, angles, and curvatures between the different components of the spine. This may be done directly from the 3D slices or as pixel distance in the most information image selected in the data ingestion process of Step 1 of the model development lifecycle.

Another secondary model may be a Pathology Diagnosis Model, which may be trained to take the most informative image from the slice, segmentations, and annotation results to predict the type of disease/disorder/condition of a spine. Using data from both healthy patients and patients suffering back issues, the AI model can be trained to predict the disease (e.g., herniation, spondylolisthesis, stenosis, etc.).

Another secondary model may be an Image-Based Report Generation Model can be based on diagnosis, measurements, and semantic/anatomical segmentations, while another AI language model can be used to author a report written in natural language. For example, the output of the AI model would be Patient X's sagittal lumbar spine image from slice 7, which showed the following measurements—L1 (x cm by y cm), L2 (x cm, y cm) . . . the curvature between LA and L5 is significantly more than normal.

An additional secondary model may be a Procedure Recommendation Model, which can be built based on training samples of patients with similar conditions and history of treatments. The model may find patients with similar pathology inferred from the imagery and what procedures the doctors and insurance companies recommended. The procedure recommendation model may use results from the explainable measurements model, semantic segmentation, and anatomical segmentation as input to generate a probability distribution over a set of procedures conducted in the past on other patients. This recommendation engine is similar to how “GOOGLE/AMAZON recommends,” e.g., “people also bought this camera item and bought this battery,” but on spinal data.

Some embodiments of the systems and methods for improving patient outcomes for musculoskeletal care can consist of the processes for the prevention of dissemination of private patient information related to their disease states. For example, in the US, HIPPA regulations require the protection of such data. Such processes can consist of the following sub-systems. These sub-systems may create HIPPA-protected firewalls.
1) Data
   a) Landing zone
   b) Curation zone
   c) Production zone
2) Model
   a) Experimental zone
   b) Production zone
   c) Explainability zone
3) API
   a) Data access internal and external
   b) Model Playbook
   c) Authentication and automation
4) User experience
   a) Physician Dashboard
   b) Insurance dashboard

Data within a landing zone may be provided with the purpose of storing raw files from PACS systems. Such data may come in and contain EHR data, image data, patient crosswalk data, or other patient-identifiable information. The data can be organized to provide API access to files in the landing zone by patient, file name, cohort, surgeon, hospital, etc. A data curation zone can be where the following happens: quality control, PHI removal, labeling, provenance tagging, AI model development (DevOps), AI model tracking (MLOps), and AI Experiments to satisfy explainability criteria. The data in this zone may allow API access to images and data by pathology, feature, semantic, and anatomical labels. A data production zone can be an area that has the plan-of-record datasets and analysis results staged to deliver to both physicians and insurance companies. API access to clean quality patient data with AI model results tagged can also be included.

A model experimentation zone can be a playground where building models occur (using the process described supra). Such an area may also include the development of a custom labeling app specific to spinal images. The collection of semantic and anatomical annotations in this area may transpire by running labeling campaigns. Labeling campaigns can occur for both normal imagery and patients with specific spinal conditions that require a procedure. Models may be built using PyTorch® and Tensorflow® AI frameworks. A model production zone can consist of a repository of approved models. The model playbook in FIG. 7 may run in production here. In some embodiments of the models within the repository, data may be tagged appropriately to every new image, no matter the source (physician, hospital, or other known source), and set up to stage the results for the explainability step. The decision framework can be implemented as intelligence that knows when to use which models for which modality, view, etc. An explainability zone can be where experiments for explainability guarantees are conducted. Models may be verified and validated for accuracy, precision, recall, and explainability. Ratings, results and explainability features/metrics can be generated per patient and saved into a database that can be served by using APIs.

APIs can be a RESTful server for getting and posting requests. Any dataset in the data zone is architectured to be accessible via easy-to-use and integrated programming interfaces. Such APIs can be implemented using Python® flash virtual environments, react.js microservice hosting.

User experience (UX) can be user interfaces run off of data and model production zones with APIs used in calls between the UI/dashboards and data sources (both internal and external PACS systems). The UX for the physician can be inspired by an open-source OHIF viewer. The insurance dashboard can be a single-page JavaScript application. Any interaction via the UI may be configured to kickstart a workflow of API GET and POST requests.

Imaging Tests and Imaging Procedures can be a type of test or procedure that makes detailed pictures of areas inside the body. Imaging tests can use different forms of energy, such as x-rays (high-energy radiation), ultrasound (high-energy sound waves), radio waves, and/or radioactive substances. They may be used to help diagnose disease, plan a treatment, or find out how well a treatment is working. Examples of imaging tests are computed tomography (CT), mammography, ultrasonography, magnetic resonance imaging (MRI), and nuclear medicine tests.

Study/Imaging events can be interchangeable with terms such as ‘study’ and ‘imaging event’ and can be considered synonymous. They can refer to a patient getting a medical imaging procedure/test performed (e.g., MRI, CT Scan, X-Ray, PET, etc.). Typically, multiple images are produced from the imaging procedure/test. The images produced are reviewed by a physician or radiologist. It's important to note that a case/study/imaging event may contain multiple “series” of images. In other words, an MRI can be a study that contains multiple series. Each series can contain multiple images.

A series can be a given sequence and alignment of images. For instance, a T2 (sequence) weighted Sagittal (alignment) series. Another example would be a STIR (sequence) axial (alignment) series. Many imaging facilities will number the various series for ease of reference.

An image can be a singular image in a given series. For example, a common description can be similar to “as image #13 of 20 in series 2.” In other words, this may refer to the 13th image (or “cut”) in the T2 sagittal series.

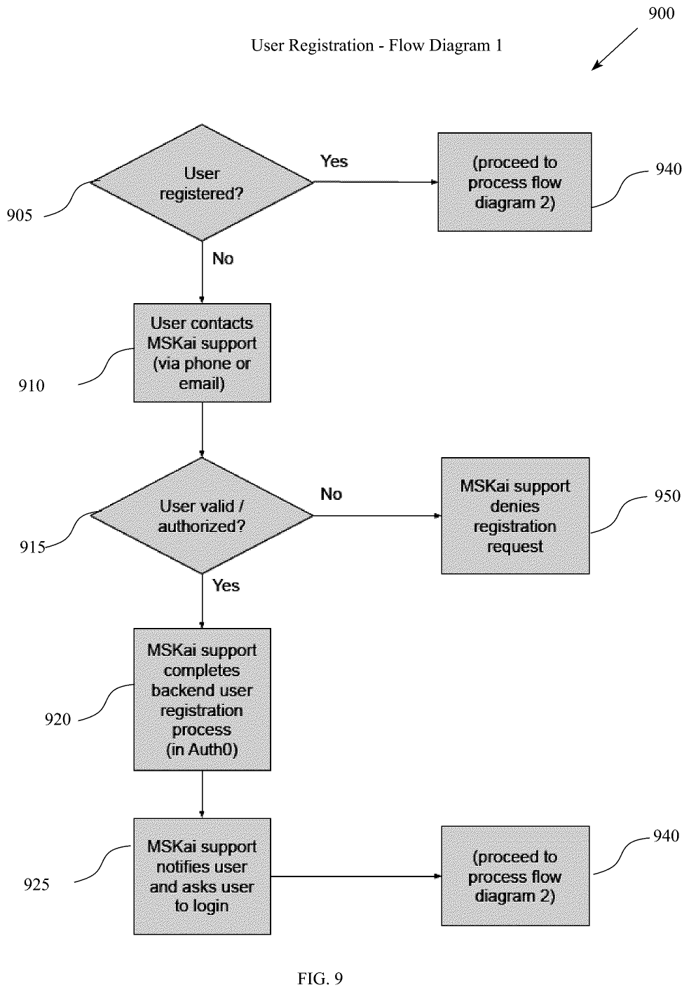

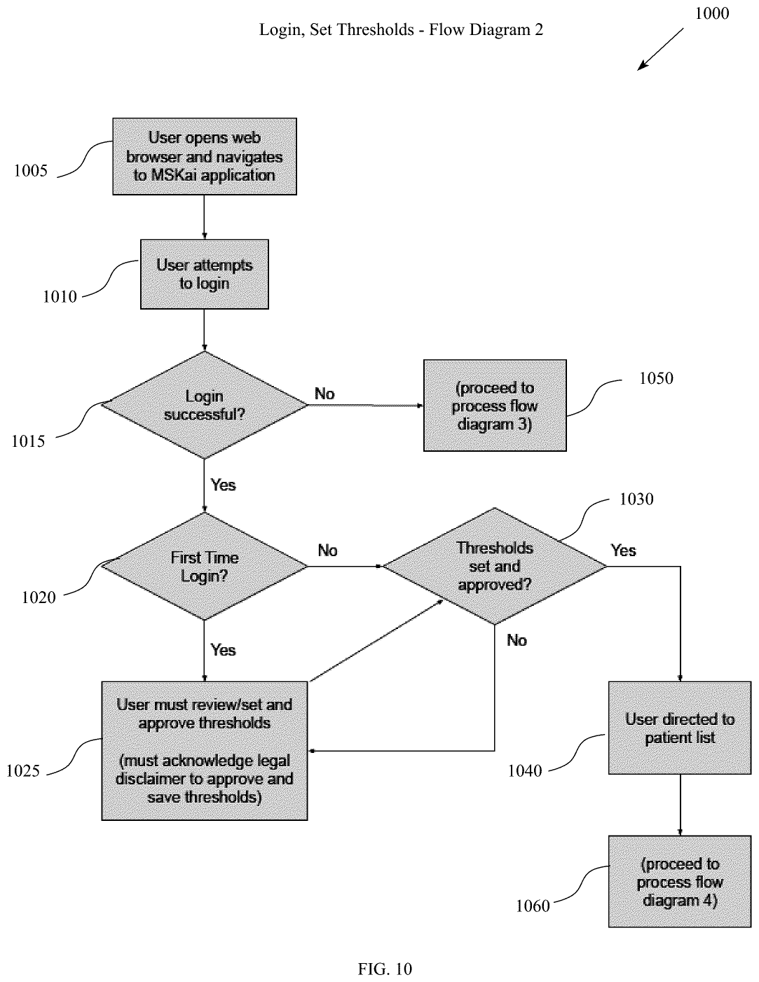

Regarding FIGS. 9-14, where an example embodiment (here being named MSKai) of a method that can utilize the model playbooks and the created annotated images as described supra. FIG. 9 illustrates a flow process, specifically flow diagram 1, providing the initiation of a user starting out with the MSKai process and their progression to flow diagram 2, illustrated in FIG. 10. Process 900 illustrates an embodiment of a user registration process where a user is either registered or denied depending on their validity as an authorized user.

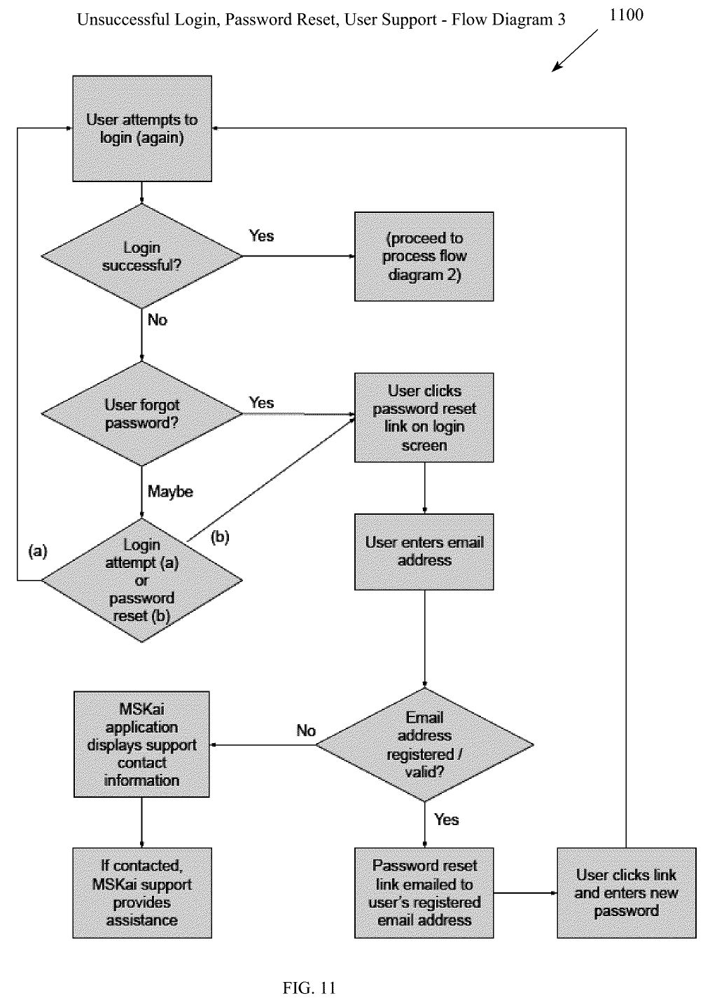

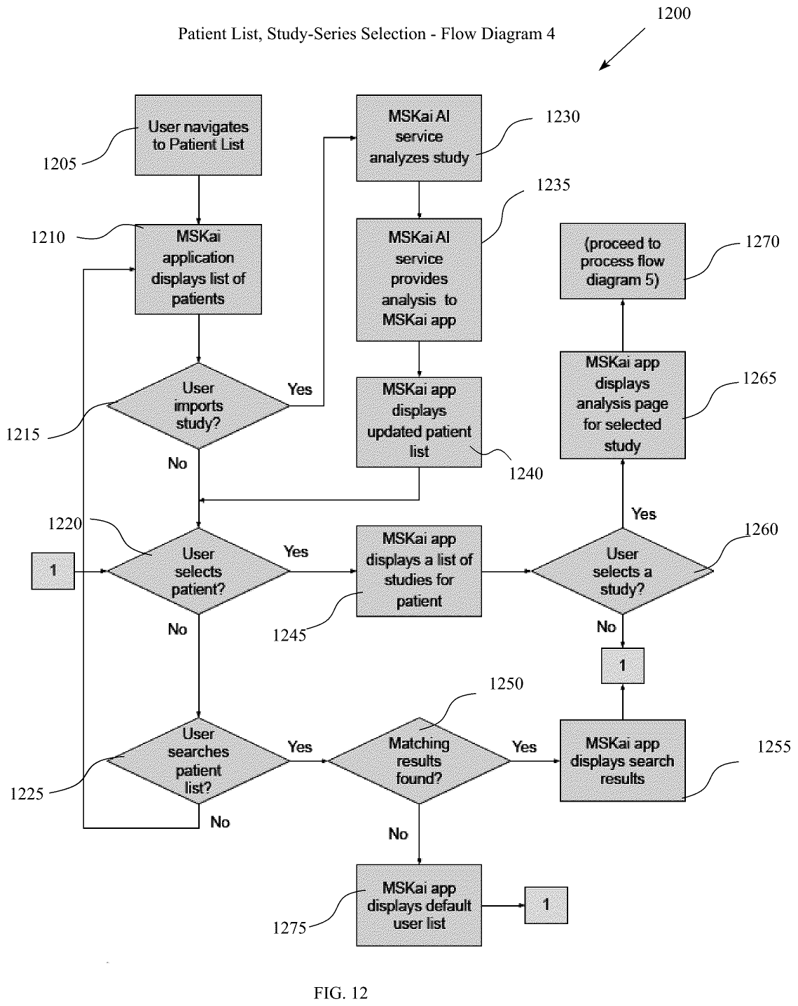

Once registered via process 900, a user can utilize process 1000, where an application (here named the MSKai Application) can be accessed via a web browser on a computer with internet access. The application can be configured to let a user use their login credentials, which were provided in process 900, to access the application. Within process 1000, if the user is successful with their login credentials and it's the user's first successful attempt, default thresholds are presented for review and approval. Default thresholds are pre-loaded values based on best medical practices. If the user chooses, threshold values can be modified to align with the users' professional experience and judgement. In all cases, the user is required to approve threshold values, both default and modified. Once threshold approval is established and the user is successful with their login credentials, they may be able to proceed to process 1200 of FIG. 12, otherwise they may be directed to process 1100 of FIG. 11. Process 1100 can be utilized by a user to reestablish their login credentials.

Process 1200, illustrated in FIG. 12, is a further description of the web application embodiment method where an application can be configured to present 1205 a user with a list of patients; from there, the user can import 1215 a study of patient images. Users may be able to scroll through images in a selected series via a mouse wheel or navigation bar. Once a study has been imported 1215, the selected images may then be algorithmically analyzed in steps 1230 and 1235. If more than one image has a highest calculated value for a sub-measurement, the image that has the highest accuracy rating as calculated by an AI model will be used.

Once analyzed, the web application embodiment in process 1200 may provide analyzed images 1265 that can be displayed side by side, where sagittal images may be on the left and Axial images may be on the right. The axial and sagittal images can be linked to improve analysis efficiency. Segmentations and label overlays can be toggled on or off as a user preference. Additionally, a table can include a pathology (measurement) name and an indication of threshold comparison results. Furthermore, tables may indicate sub-measurement report language for pathology analysis; e.g., they may reflect sub-measurement value comparisons to threshold parameters. Users may also have the ability to modify their thresholds. Once modified, a user can be required to acknowledge the change and agree to a legal statement.

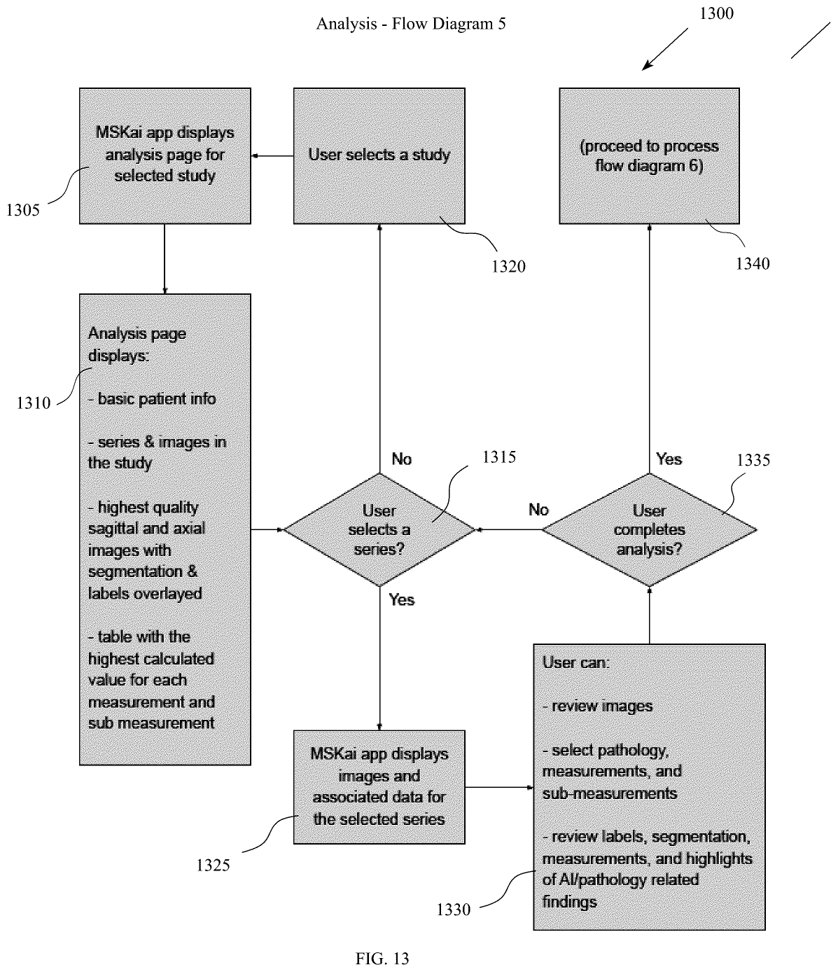

The analysis is continued in FIG. 13, where flow diagram 5 includes process 1300. The web application embodiment can be configured to provide a display analysis page 1310 having a series of data that can include basic patient info, the series & images in the study, the highest quality sagittal and axial images with segmentation that may include labels overlayed, and a table with the highest calculated value for each measurement and sub-measurement. The user may then be able to review images, select pathology, measurements, and sub-measurements. A user may also be able to review labels, segmentation, measurements, and highlights of AI/pathology-related findings.

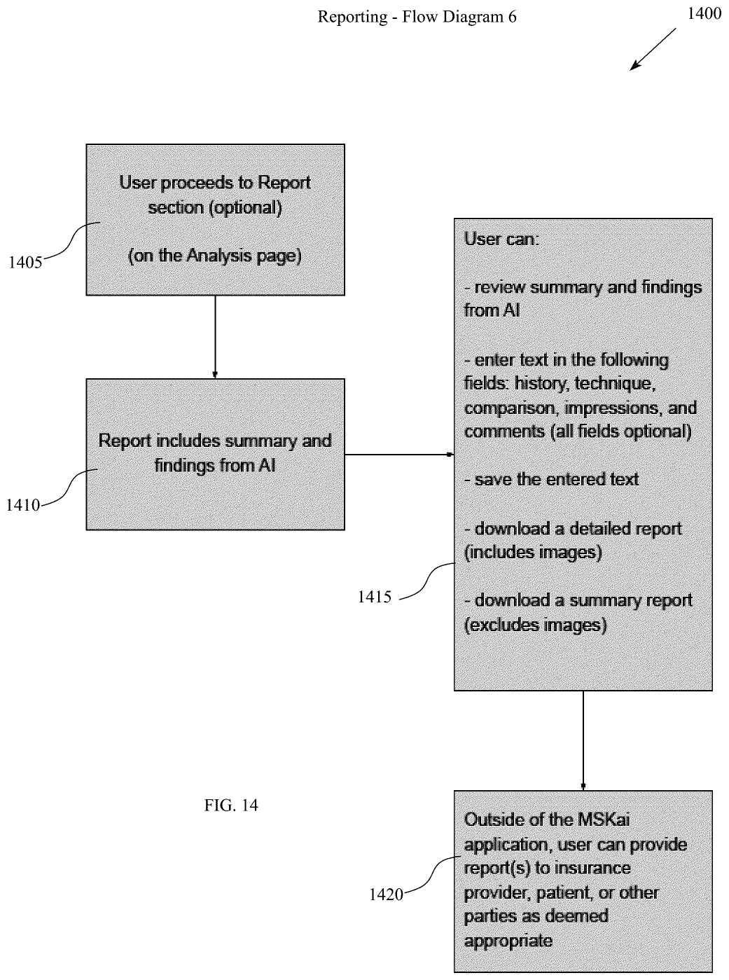

Once the review process is completed, a user may then use the web application embodiment to generate a report, as illustrated in FIG. 14, flow diagram 6, which includes process 1400. The web application embodiment may be configured to let the user review summary and findings from the AI model results, enter text into fields (fields can include history, technique, comparison, impressions, and comments (all fields can be optional)), save entered text, download a detailed report, and download a summary report. All the reports may include or exclude desired images. Outside of the web application embodiment, the user can provide a report(s) to an insurance provider, patient, or other parties as deemed appropriate.

Persons of ordinary skill in arts relevant to this disclosure and subject matter hereof will recognize those embodiments may comprise fewer features than illustrated in any individual embodiment described by example or otherwise contemplated herein. Embodiments described herein are not meant to be an exhaustive presentation of ways in which various features may be combined and/or arranged. Accordingly, the embodiments are not mutually exclusive combinations of features; rather, embodiments can comprise a combination of different individual features selected from different individual embodiments, as understood by persons of ordinary skill in the relevant arts. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is also intended to include features of a claim in any other independent claim, even if this claim is not directly made dependent on the independent claim.

We claim:

1. A method for improving identification of pathologies for musculoskeletal patients comprising the following steps:
providing a computing unit configured to utilize an algorithm trained with an AI engine using images of musculoskeletal pathology;
obtaining at least one patient image;
applying the algorithm to the patient image to identify a pathology;
applying an application, stored within the computing unit, to annotated anatomical measurement within the algorithmically analyzed patient image; and
generating a report comprising an annotated patient image.

2. The method for improving identification of pathologies for musculoskeletal patients of claim 1, wherein the AI engine is either a knowledge-centric intelligence model, or a neural network model.

3. The method for improving identification of pathologies for musculoskeletal patients of claim 1, wherein after the at least one patient image is obtained a further step follows of analyzing the at least one patient image by a modality detector.

4. The method for improving identification of pathologies for musculoskeletal patients of claim 2, wherein the knowledge-centric intelligence model is configured to generate an explanation of a decision by the algorithm.

5. The method for improving identification of pathologies for musculoskeletal patients of claim 2, wherein a decision from the neural network model is validated with a feature-measurement model.

6. The method for improving identification of pathologies for musculoskeletal patients of claim 2, wherein a 3D model outputs an overlay of physics-based measurements and algorithmic predictions.

7. The method for improving identification of pathologies for musculoskeletal patients of claim 3, wherein after the at least one patient image is analyzed by the modality detector a further step follows of analyzing the at least one patient image by at least one secondary model.

8. A system for generating annotated patient images comprising:
a computing unit configured to utilize an algorithm trained with an AI engine using images of musculoskeletal pathology;
at least one patient image, wherein the algorithm is applied to the patient image to identify a pathology;
an application, stored within the computing unit, wherein the application is configured to annotated anatomical measurement within the algorithmically analyzed patient image; and
wherein the computing unit is configured to generate a report comprising the annotated patient image.

9. The system for generating annotated patient images of claim 8, wherein the AI engine is either a knowledge-centric intelligence model, or a neural network model.

10. The system for generating annotated patient images of claim 8, wherein the computing unit further comprises a modality detector configured to analyze the at least one patient image.

11. The system for generating annotated patient images of claim 8, wherein the AI engine is either a knowledge-centric intelligence model, or a neural network model.

12. The system for generating annotated patient images of claim 8, wherein the computing unit further comprises a modality detector configured to analyze the at least one patient image.

13. The system for generating annotated patient images of claim 9, wherein the knowledge-centric intelligence model is configured to generate an explanation of a decision by the algorithm, wherein the explanation is added to the report.

14. The system for generating annotated patient images of claim 9, wherein the computing unit further comprises a feature-measurement model, wherein the feature-measurement model is configured to validate a decision from the neural network model.

15. The system for generating annotated patient images of claim 9, wherein a 3D model outputs an overlay of physics-based measurements and algorithmic predictions.

16. The system for generating annotated patient images of claim 10, further comprising at least one secondary model, wherein the at least one secondary model is configured to receive and analyze the at least one patient image from the modality detector.

17. The system for generating annotated patient images of claim 8, wherein the report is provided via a web-based application.
